(19) United States
(12) Patent Application Publication
(10) Pub. No.: US 2024/0167466 A1
(43) Pub. Date: May 23, 2024

(54) PERISTALTIC PUMP EQUIPPED WITH A RELEASE SYSTEM TO DISENGAGE THE FLUID TUBE

(71) Applicants: BAXTER HEALTHCARE SA, Glattpark (Opfikon) (CH); BAXTER INTERNATIONAL INC., Deerfield, IL (US)

(72) Inventor: Marco PARALUPPI, Medolla, Modena (IT)

(21) Appl. No.: 18/283,418

(22) PCT Filed: Mar. 9, 2022
(86) PCT No.: PCT/EP2022/056101

§ 371 Date: Sep. 21, 2023

(30) Foreign Application Priority Data
Mar. 25, 2021 (IT) 102021000007358

(51) Int. Cl. F04B 43/12

(52) U.S. Cl. CPC F04B 43/1246

(57) ABSTRACT

Disclosed herein is a peristaltic pump configured to receive a fluid line and comprising a stator having a pump race, a rotor surrounded by the pump race and configured to rotate about a rotor axis. The rotor comprises a contrasting element configured to abut against the fluid line. The contrasting element is movable between a released position where the contrasting element is away from the pump race, and an operative position where the at least one contrasting element is approached to the pump race and configured to abut against the fluid line. In the released position, the contrasting element is at a distance from the pump race higher than in the operative position. The peristaltic pump further comprises a releasing member borne by the stator and movable between an active position and a rest position, where the releasing member is spatially shifted with respect to the rotor axis.

### FIELD OF THE INVENTION

The present invention relates to a peristaltic pump having a releasable system to disengage a fluid tube. The invention also relates to a method to install and remove a fluid tube from a peristaltic pump provided with the releasable system of the present invention.

Furthermore, the invention also relates to a method to balance fluid pressures in the fluid line, upstream and downstream the peristaltic pump of the present invention.

Furthermore, the invention also relates to a method to prevent damages to a fluid line mounted on the peristaltic pump of the present invention during prolonged inactivity periods.

### BACKGROUND

Peristaltic pumps are commonly used to promote fluid flow in a fluid line: in particular peristaltic pumps are used in medical field because of safety reasons as these pumps allow to prevent the fluid from contacting external parts other than the line, thereby improving sterilization.

In common peristaltic pumps, rollers or shoes compress the flexible line as they rotate, creating a negative pressure behind that recalls the fluid, and a positive pressure forward causing the fluid to advance. Common peristaltic pumps comprise a rotor carrying the rollers and a stator carrying a pump race partially surrounding the rotor and defining a gap in between: the flexible line is operatively arranged within this gap so that the line is compressed, in an operative condition, between a roller and the external pump race. Rotation of the rotor determines advancing of the roller with respect to the race over the line, causing the fluid in the line to advance.

During operations, it may be requested to periodically remove and reinstall the fluid line between the race and the rollers. Removal and reinstallation procedures imply to squeeze and forcibly manipulate the fluid line, as the latter is compressed or has to be compressed between the rollers and the surrounding race, thereby stressing repetitively the fluid line: manipulation may lead to premature damage of the fluid line causing the need of replacing it and negatively affecting the safety level of the pump system. Notably, a damage occurring to a blood line, such as a crack on the line, during an extracorporeal blood treatment, may lead to extremely dangerous consequences for the patient, such as massive blood loss or allowing bacteria to enter the blood stream.

Furthermore, as the fluid line is compressed between the rollers and the track, installation and removal procedures may result to be challenging, thereby requiring the intervention of a professional person, i.e. a nurse or a doctor, and extending the time required to accomplish the task.

### Scope of the Invention

The scope of this invention is therefore to at least partially solve one or more of the drawbacks and/or limitations of the prior art solutions.

A first scope is to provide a peristaltic pump able to substantially reduce or completely remove the risk of damaging the fluid line during periodical installation and removal procedures.

A further scope is to provide a peristaltic pump able to ease periodical installation and removal procedures of a fluid line without requiring massive manipulation.

A further scope is to provide a peristaltic pump combining manufacturing costs low and high reliability with the preceding features.

These scopes and more, which will appear more from the following description, are substantially achieved by a peristaltic pump in accordance with one or more of the following claims and/or aspects.

### Summary Section

A first aspect refers to a peristaltic pump (1) configured to receive a fluid line (50), said peristaltic pump comprising:
a stator (2) comprising a pump race (3);
a rotor (10) surrounded at least partially by the pump race (3) and configured to rotate about a rotor axis (RA), said rotor (10) comprising:
   at least one contrasting element (11) configured to abut against the fluid line (50), the at least one contrasting element (11) being movable towards and away with respect to the rotor axis (RA) and the pump race (3) between:
      a released position wherein the at least one contrasting element (11) is away from the pump race (3), and
      an operative position wherein the at least one contrasting element (11) is approached to the pump race (3) and configured to abut against the fluid line (50), in the released position the at least one contrasting element (11) being at a distance from the pump race (3) higher than in the operative position,
   a thrusting element (20) configured to act, at least in the operative position, in thrust on said contrasting element (11) in an outwardly direction towards the pump race (3);
a releasing system (30) comprising a releasing member (31) borne by the stator (2) and movable between an active position and a rest position, the releasing member (31) being spatially shifted with respect to the rotor axis (RA).

In a 2^{nd} aspect according to the preceding aspect, the peristaltic pump (1) is configurable in at least:
an operative configuration wherein the releasing member (31) is moved in the rest position to allow the at least one contrasting element (11) to move in the operative position, the contrasting element (11) being thrusted by the thrusting element (20) in the outwardly direction;
a released configuration wherein the releasing member (31) is moved in the active position, said releasing member (31) engaging the contrasting element (11) and moving the contrasting element (11) in the released position away from the pump race (3).

A 3^{rd} aspect is directed to a method (1000) to switch the peristaltic pump (1) from the operative configuration to the released configuration and vice versa, said peristaltic pump (1) being optionally according to any one of the preceding aspects, wherein the method to switch the peristaltic pump (1) from the operative configuration to the released configuration comprises at least the following steps:
(1001) arresting or rotating the rotor (10) to a predefined angular unlocking position with respect to the stator (2), the predefined angular unlocking position being defined as a position wherein at least one of the contrasting elements (11) is engageable by the releasing member (31) to switch from the operative position to the released position and vice versa;
(1002) moving the releasing member (31), in particular rotating a releasing cam (32), from the rest position to the active position to move the at least one contrasting element (11) from the operative position to the released position, said step of moving the releasing member (31) being performed manually by an operator or by a control unit (80) through an actuator;

and wherein the method to switch the peristaltic pump (1) from the released configuration to the operative configuration comprises at least the following steps:
arresting or rotating the rotor (10) to a predefined angular unlocking position with respect to the stator (2), the predefined angular unlocking position being defined as a position wherein at least one of the contrasting elements (11) is engageable by the releasing member (31) to switch from the operative position to the released position and vice versa;
(1005) moving the releasing member (31), in particular rotating the releasing cam (32), from the active position to the rest position to move the at least one contrasting element (11) from the released position to the operative position, said step of moving the releasing member (31) being performed manually by an operator or by the control unit (80) through an actuator.

In a 4^{th} aspect according to any one of the preceding aspects:
the releasing member (31) is a releasing cam (32) rotatable about a cam axis (CA), said cam axis being spatially shifted with respect to the rotor axis (RA), and wherein:
   in the operative configuration, the releasing cam (32) is rotated in the rest position to allow the at least one contrasting element (11) to remain in the operative position, the contrasting element (11) being thrusted by the thrusting element (20) in the outwardly direction;
   in the released configuration, the releasing cam (32) is rotated in the active position, said releasing cam (32) engaging the at least one contrasting element (11) and maintaining the at least one contrasting element (11) in the released position away from the pump race (3);

and/or
the releasing member (31) is a translating member movable by translation along a rectilinear or curvilinear path, wherein:
   the operative configuration, the translating member is moved in the rest position to allow the at least one contrasting element (11) to remain in the operative position, the contrasting element (11) being thrusted by the thrusting element (20) in the outwardly direction;
   in the released configuration, the translating member is moved in the active position, said translating member engaging the at least one contrasting element (11) and maintaining the at least one contrasting element (11) in the released position away from the pump race (3).

In a 5^{th} aspect according to any one of the preceding aspects, the cam axis (CA) of the releasing cam (32) is fixed with respect to the stator (2) and wherein the releasing cam (32) is configured to rotate about the cam axis relative to the stator (2).

In a 6^{th} aspect according to any one of the preceding aspects, the cam axis (CA) is substantially parallel to the rotor axis (RA).

In a 7^{th} aspect according to any one of the preceding aspects, the releasing member (31), in particular the releasing cam (32) and more in particular the cam axis (CA) of the releasing cam (32), is interposed between the rotor axis (RA) and the pump race (3) with respect to a radial direction normal to the rotor axis (RA).

In a 8^{th} aspect according to any one of the preceding aspects, the releasing member (31), in particular the releasing cam (32) and more in particular the cam axis (CA) of the releasing cam (32), is closer to the pump race (3) than to the rotor axis (RA).

In a 9^{th} aspect according to any one of the preceding aspects, the stator (2) comprises a base wall (2a), wherein the pump race (3) protrudes in height from said base wall (2a) defining an internal volume confined in depth by the base wall (2a) and laterally by the pump race (3), and wherein the rotor (10) is arranged at least partially within said internal volume of the stator (2).

In a 10^{th} aspect according to any one of the preceding aspects, the releasing cam (32) includes a bearing axle (32a) rotatable about the cam axis (CA), and a cam-shaped top element (32b) fixed at an end of to the bearing axle (32a), the bearing axle (32a) in combination with the cam-shaped top element (32b) defining the releasing cam (32) as a single body.

In a 11^{th} aspect according to any one of the preceding two aspects, the base wall (2a) of the stator (2) comprises a through hole (4) allowing the bearing axle (32a) of the releasing cam (32) to pass through and enter the internal volume.

In a 12^{th} aspect according to any one of the preceding two aspects, the cam-shaped top element (32b) of the releasing cam (32) emerges in height from said base wall (2a) of the stator (2) into the internal volume defining a cam-shaped lateral abutment wall (32c).

In a 13^{th} aspect according to any one of the preceding three aspects:
when the releasing cam (32) is in the active position, said cam-shaped lateral abutment wall (32c) abuts against the at least one contrasting element (11) of the rotor (10) to define the released configuration of the peristaltic pump, and
when the releasing cam (32) is in the rest position, said cam-shaped lateral abutment wall (32c) allows the at least one contrasting element (11) to remain in the operative position to define the operative configuration of the peristaltic pump.

In a 14^{th} aspect according to any one of the preceding aspects, the pump race (3) has a “C” or “U” shape, e.g., semicircular shape, substantially concentric to the rotor axis (RA), and wherein the peristaltic pump is configured to receive the fluid line (50) between the pump race (3) and the contrasting element (11) defining a pump loop portion.

In a 15^{th} aspect according to any one of the preceding aspects, when the pump is in the operative configuration, the contrasting element (11) is configured to compress the fluid line (50) against the pump race.

In a 16^{th} aspect according to any one of the preceding aspects, the rotor (10) comprises a rotor plate (10a) bearing the at least one contrasting element (11), and wherein each contrasting element (11) includes:
at least one roller or shoe (12) configured to contact and squeeze the fluid line (50) against the pump race (3) in the operative condition of the peristaltic pump;
a lever arm (13) extending between a hinged end (13a) and a free end (13b), said lever arm (13) being rotatable about the hinged end (13a) about a hinge axis (HA), wherein:
   the hinged end (13a) is hinged to said rotor plate (10a) thereby allowing rotation of the lever arm (13) about the hinge axis (HA), and
   the free end (13b) carries the at least one roller or shoe (12), wherein a rotation of the lever arm (13) about the hinge axis (HA) determines the passage of the respective contrasting element (11) between the operative position and the released position.

In a 17^{th} aspect according to any one of the preceding aspects, the peristaltic pump comprises two or more contrasting elements (11) substantially diametrically opposite each other with respect to the rotor axis (RA).

In a 18^{th} aspect according to any one of the preceding aspects, the peristaltic pump comprises three or more contrasting elements (11) angularly spaced each other in a substantially evenly manner, in particular the three or more contrasting element being equally spaced each other.

In a 19^{th} aspect according to any one of the preceding aspects, the contrasting elements (11) comprise two or more contrasting elements (11), the releasing member (31) being configured to engage one contrasting element at a time between the two or more contrasting elements (11).

In a 20^{th} aspect according to any one of the preceding aspects, the contrasting elements (11) comprise three or more contrasting elements (11), the releasing member (31) being configured to engage:
two contrasting elements at a time between the three or more contrasting elements (11); or
one contrasting element at a time between the three or more contrasting elements (11).

In a 21^{st} aspect according to any one of the preceding aspects from 16 to 20, the lever arm (13) comprises an extended body and an abutment protrusion (14) emerging from said extended body, said abutment protrusion (14) being configured to abut against the cam-shaped lateral abutment wall (32c) of the releasing cam (32) when the releasing cam is in the active position.

In a 22^{nd} aspect according to the preceding aspect, the abutment protrusion (14) is interposed between said extended body and the base wall (2a) of the stator (2).

In a 23^{rd} aspect according to any one of the preceding aspects, the thrusting element (20) comprises:
at least one elastic element (21), in particular a spring or a rubber-like element, more in particular a compressive or traction spring, acting on the at least one contrasting element (11), in particular on each contrasting element, and configured to move the at least one contrasting element (11) in the outwardly direction, said at least one elastic element allowing, in particular causing, movement of the at least one contrasting element (11) from the released position to the operative position;

or
a thrusting cam (22) movable by rotation between:
   a thrusting position wherein the thrusting cam (22) abuts against the at least one contrasting element (11) causing the contrasting element (11) to move in the operative position, and
   a non-thrusting position wherein the thrusting cam (22) allows the contrasting element (11) to move in the released position.

In a 24^{th} aspect according to any one of the preceding aspects, the elastic element is configured to:
exert, when the pump is in the operative configuration, a force on the at least one contrasting element (11) such that the latter is able to adequately squeeze, in particular deform, the fluid line (50) to promote fluid flow, and
deform itself to allow the at least one contrasting element (11) to move from the operative position to the released position.

In a 25^{th} aspect according to any one of the preceding aspects, the releasing member (31), in particular the releasing cam (32), is configured, during a passage between the operative configuration to the released configuration, to exert a force on the at least one contrasting element (11) opposite in direction to a force exerted by the thrusting element (20) on the at least one contrasting element (11).

In a 26^{th} aspect according to any one of the preceding aspects, the peristaltic pump comprises a drive member (40) configured to cause rotation of the rotor about the rotor axis (RA), said drive member being an electric motor configured to rotate the rotor to promote fluid flow within the fluid line (50).

In a 27^{th} aspect according to any one of the preceding aspects, the rotor comprises a rotor axle (10b), and the base wall (2a) of the stator comprises an through aperture (6) coincident with the rotor axis (RA), said through aperture (6) allowing the rotor axle (10b) of the rotor (10) to pass through and defining an aperture axis (AA), said aperture axis being spatially shifted with respect to the releasing member, in particular the aperture axis (AA) being substantially parallel to the cam axis (CA) of the releasing cam.

In a 28^{th} aspect according to any one of the preceding aspects, the stator (2), in particular the base wall (2a) and the pump race (3), is/are made of plastic material.

In a 29^{th} aspect according to any one of the preceding aspects, when the pump switches between the operative configuration and the released configuration, the rotor (10) is arranged in a predefined angular unlocking position.

In a 30^{th} aspect according to the preceding aspect, when the rotor (10) is in said predefined angular unlocking position, at least one of the contrasting elements (11) is engageable by the releasing member (31) to switch from the operative position to the released position and vice versa.

In a 31^{st} aspect according to any one of the two preceding aspects, when the rotor (10) is in said predefined angular unlocking position, only one contrasting element (11) of the two or three or more contrasting elements (11) is engageable by the releasing member (31) to switch from the operative position to the released position and vice versa.

In a 32^{nd} aspect according to any one of the three preceding aspects, in said predefined angular unlocking position, the cam-shaped lateral abutment wall (32c) of the releasing cam (32) faces the abutment protrusion (14) of the lever arm (13) of one respective contrasting element (11).

In a 33^{rd} aspect according to any one of the four preceding aspects, in said predefined angular unlocking position the releasing cam (32) is interposed between the abutment protrusion (14) of the lever arm (13) and the pump race (3) with respect to a direction normal to the rotor axis (RA).

In a 34^{th} aspect according to any one of the preceding aspects, the pump race (3) has:
an inner wall configured to contact the fluid line, said inner wall defining an operative angular arch for the contrasting elements, said contrasting elements facing said inner wall of the pump race, and
a lateral aperture configured to allow an inlet tract of the fluid line (50) to be received by the pump, and an exit tract of the fluid lined to exit the pump, said lateral aperture defining a non-operative angular arch for the contrasting elements, in particular the contrasting elements when in said non-operative angular arch are not configured to fully cooperate with the fluid line (50).

In a 35^{th} aspect according to the preceding aspect, the rotor (10), when arranged in the predefined angular unlocking position, has:
at least one first contrasting element positioned in the operative angular arch, wherein said at least one first contrasting element is engageable by the releasing member (31); and optionally
at least one second contrasting element positioned in said non-operative angular arch, wherein said at least one second contrasting element is not engageable by the releasing member (31).

In a 36^{th} aspect according to the preceding aspect, the releasing member 31, when passing from the rest position to the active position, is configured to engage said at least one first contrasting element to move the latter in the released position.

In a 37^{th} aspect according to any one of the preceding aspects, the releasing member 31, when passing from the rest position to the active position, is configured not to engage said at least one second contrasting element.

In a 38^{th} aspect according to any one of the preceding aspects, the peristaltic pump comprises:
a drive motor (60) operatively connected to the rotor (10) and configured to rotate said rotor about the rotor axis (RA) to determine a fluid flow in the fluid line (50);
a position sensor (70) configured to emit a signal representative of an angular position of the rotor (10) with respect to the stator (2), said position sensor being in particular a Hall sensor configured to detect a position or a passage of the roller or shoe (12);
a control unit (80) operatively connected to the drive motor (60) and the position sensor (70), the control unit being configured to receive the signal emitted by the position sensor (70) and to determine an angular position of the rotor with respect to the stator (2).

In a 39^{th} aspect according to any one of the preceding aspects, the control unit is configured to perform a:
releasing procedure configured to switch the peristaltic pump from the operative configuration to the released configuration, said releasing procedure comprising:
   arresting or rotating the rotor (10) to a predefined angular unlocking position based on the position detected by the position sensor (70);
   moving the releasing member (31), in particular rotating the releasing cam (32), from the rest position to the active position to move the at least one contrasting element (11) from the operative position to the released position, said step of moving the releasing member (31) being performed manually by an operator or by the control unit (80) through an actuator;
   removing the fluid line (50) between the at least one contrasting element (11) and the pump race (3).

In a 40^{th} aspect according to any one of the preceding aspects, the control unit is configured to perform:
a setting procedure, in particular subsequent to the releasing procedure, configured to switch the peristaltic pump from the released configuration to the operative configuration, said setting procedure comprising:
   arresting or rotating the rotor (10) to a predefined angular unlocking position based on the position detected by the position sensor (70);
   moving the releasing member (31), in particular rotating the releasing cam (32), from the rest position to the active position to move the at least one contrasting element (11) from the operative position to the released position, said step of moving the releasing member (31) being performed manually by an operator or by the control unit (80) through an actuator;
   installing the fluid line (50) between the at least one contrasting element (11) and the pump race (3)
   moving the releasing member (31), in particular rotating the releasing cam (32), from the active position to the rest position to move the at least one contrasting element (11) from the released position to the operative position, said step of moving the releasing member (31) being performed manually by an operator or by the control unit (80) through an actuator.

In a 41^{st} aspect according to any one of the preceding aspects, the predefined angular unlocking position is defined as a position wherein at least one of the contrasting elements (11) is engageable by the releasing member (31) to switch from the operative position to the released position and vice versa.

In a 42^{nd} aspect according to any one of the preceding aspects, the cam-shaped lateral abutment wall (32c) of the releasing cam (32) faces the abutment protrusion (14) of the lever arm (13) of one respective contrasting element (11), in said predefined angular unlocking position the releasing cam (32) is interposed between the abutment protrusion (14) of the lever arm (13) and the pump race (3).

In a 43^{rd} aspect according to any one of the preceding aspects, the step of the setting and/or releasing procedure of arresting or rotating the rotor (10) comprises the step of commanding the drive motor (60) to position the rotor (10) at the predefined angular unlocking position.

In a 44^{th} aspect according to any one of the preceding aspects, the control unit (80) is configured to switch the peristaltic pump (1) from the operative configuration to the released configuration by performing at least the following steps:
arresting or rotating the rotor (10) at a predefined angular unlocking position with respect to the stator (2), the predefined angular unlocking position being defined as a position wherein at least one of the contrasting elements (11) is engageable by the releasing member (31) to switch from the operative position to the released position and vice versa;
moving the releasing member (31), in particular rotating a releasing cam (32), from the rest position to the active position to move the at least one contrasting element (11) from the operative position to the released position, said step of moving the releasing member (31) being performed manually by an operator or by a control unit (80) through an actuator.

In a 45^{th} aspect according to any one of the preceding aspects, the control unit (80) is configured to switch the peristaltic pump (1) from the released configuration to the operative configuration by performing at least the following steps:
arresting or rotating the rotor (10) to a predefined angular unlocking position with respect to the stator (2), the predefined angular unlocking position being defined as a position wherein at least one of the contrasting elements (11) is engageable by the releasing member (31) to switch from the operative position to the released position and vice versa;
moving the releasing member (31), in particular rotating the releasing cam (32), from the active position to the rest position to move the at least one contrasting element (11) from the released position to the operative position, said step of moving the releasing member (31) being performed manually by an operator or by the control unit (80) through an actuator.

In a 46^{th} aspect according to any one of the preceding aspects, the control unit is configured to perform a substituting procedure configured to allow replacing the fluid line, said substituting procedure comprising:
arresting or rotating the rotor (10) to a predefined angular unlocking position based on the position detected by the position sensor (70);
moving the releasing member (31), in particular rotating the releasing cam (32), from the rest position to the active position to move the at least one contrasting element (11) from the operative position to the released position, said step of moving the releasing member (31) being performed manually by an operator or by the control unit (80) through an actuator;
removing the fluid line (50) between the at least one contrasting element (11) and the pump race (3);
installing the fluid line (50) between the at least one contrasting element (11) and the pump race (3)
moving the releasing member (31), in particular rotating the releasing cam (32), from the active position to the rest position to move the at least one contrasting element (11) from the released position to the operative position, said step of moving the releasing member (31) being performed manually by an operator or by the control unit (80) through an actuator.

In a 47^{th} aspect according to the preceding aspect, the step of the substituting procedure of arresting or rotating the rotor (10) comprises the step of commanding the drive motor (60) to position the rotor (10) to the predefined angular unlocking position.

In a 48^{th} aspect according to any one of the preceding aspects, the control unit (80) is configured to perform a pressure balancing procedure to re-balance fluid pressures across the peristaltic pump (1), wherein the pressure balancing procedure comprises at least the steps of:
arresting, or rotating the rotor (10), to the predefined angular unlocking position based on the position detected by the position sensor (70) of the rotor (10);
moving the releasing member (31), in particular rotating the releasing cam (32), from the rest position to the active position to move the at least one contrasting element (11) from the operative position to the released position, the step of moving the releasing member (31) being performed manually by an operator or by the control unit (80) through the actuator of the releasing member (31).

In a 49^{th} aspect according to the preceding aspect, the control unit (80) is configured to:
receive at least a pressure signal representative of a fluid pressure upstream and downstream the peristaltic pump (1) or a signal representative of a differential pressure across the pump, in particular a differential pressure between an upstream section of the fluid line and a downstream section of the fluid line;
determine a differential pressure value representative of a differential pressure across the pump (1);
compare said differential pressure value with at least one respective threshold value;
if said differential pressure value exceeds the at least one respective threshold value, perform the balancing procedure.

In a 50^{th} aspect according to any one of the preceding aspects, the control unit is configured to perform a inactivity procedure configured to prevent damage to the fluid line (50) during prolonged inactivity periods of the pump (1), said inactivity procedure comprising:
arresting or rotating the rotor (10) at the predefined angular unlocking position based on the position detected by the position sensor (70);
switching the pump from the operative configuration to the released configuration in particular by moving the releasing member (31), optionally rotating the releasing cam (32), from the rest position to the active position to move the at least one contrasting element (11) from the operative position to the released position, said step of moving the releasing member (31) being performed manually by an operator or by the control unit (80) through an actuator;

wherein, during said prolonged inactivity periods of the pump (1), the fluid line (50) is continuously or permanently mounted onto the pump (1), in particular wherein the fluid line (50) is interposed between at least one of the contrasting element (11) and the pump race (3).

In a 51^{st} aspect according to the preceding aspect, the control unit (80) is configured to:
evaluate an inactivity time period within the pump (1) is inactive;
compare said inactivity time period with an inactivity time period threshold;
if the inactivity time period exceeds the inactivity time period threshold, trigger the inactivity procedure.

In a 52^{nd} aspect according to any one of the preceding aspects, the at least one contrasting element (11), when arranged in the released position, is configured to reduce or completely remove a compression force on the fluid line (50) when the latter is installed on the pump (1).

In a 53^{rd} aspect according to any one of the preceding aspects, the at least one contrasting element (11), when arranged in the released position, defines a distance with respect to the pump race (3) comprised between 0.7 and two times an external diameter of the fluid line (50), more in particular between one and 1.5 times an external diameter of the fluid line (50).

In a 54^{th} aspect according to any one of the preceding aspects, the at least one contrasting element (11), when arranged in the released position, defines a distance with respect to the pump race (3) comprised between 2.5 mm and 10 mm (more specifically between 3 mm and 8 mm), in particular said distance being measured between the roller of the contrasting element and the pump race, said distance being defined as a distance interposed between a portion of the contrasting element configured to abut against the fluid line (50) and the pump race (3).

In a 55^{th} aspect according to any one of the preceding aspects, the releasing system (30) comprises a position sensor (71) operatively connected to the control unit (80) and configured to emit a signal representative of the active or rest position of the releasing member (31), said control unit (80) being configured to selectively determine whether the releasing member (31) is in the active or rest position.

In a 56^{th} aspect according to any one of the preceding aspects, the distance between the at least one contrasting element (11) and the pump race is defined as a distance interposed between a portion of the contrasting element configured to abut against the fluid line (50) and the pump race (3).

In a 57^{th} aspect according to any one of the preceding aspects, the distance between the at least one contrasting element (11) and the pump race is defined as a distance interposed between a roller or shoe (12), when arranged in the operative arc of the pump race (3), and the pump race (3).

In a 58^{th} aspect according to any one of the preceding aspects, the pump comprises a drive motor (60) operatively connected to the rotor (10) and configured to rotate said rotor about the rotor axis (RA) to determine a fluid flow in the fluid line (50).

In a 59^{th} aspect according to any one of the preceding aspects, the pump comprises a position sensor (70) configured to emit a signal representative of an angular position of the rotor (10) with respect to the stator (2), said position sensor being in particular a Hall sensor configured to detect a position or a passage of the roller or shoe (12).

In a 60^{th} aspect according to any one of the preceding aspects, the control unit (80) is configured to:
detect the position of the rotor (10) by the position sensor (70);
evaluate whether said detected position of the rotor (10) is coincident with the predefined angular unlocking position;
if said detected position of the rotor (10) is coincident with the predefined angular unlocking position, allow the actuator of the releasing member (31) to move the releasing member from the rest position to the active position;
if said detected position of the rotor (10) does not coincide with the predefined angular unlocking position, prevent the actuator of the releasing member (31) to move the releasing member from the rest position to the active position.

In a 61^{st} aspect according to any one of the preceding aspects, the peristaltic pump comprises an actuator operatively connected to a control unit (80) and to the releasing member (31), the actuator being configured to move the releasing member (31) between the rest position and the active position upon command of the control unit (80).

In a 62^{nd} aspect according to any one of the preceding aspects, the control unit (80) is configured to:
detect the position of the releasing member (31) by the position sensor (71) of the releasing system (30);
evaluate whether said detected position of the releasing member (31) corresponds to the active position or the rest position;
if the releasing member (31) is detected to be in the active position, prevent rotation of the rotor (10) by inhibiting rotation of the drive motor (60) of the rotor (10).

In a 63^{rd} aspect according to any one of the preceding aspects, the releasing member (31) of the releasing system (30) is configured, upon movement from the rest position to the active position, to move the at least one contrasting element (11) or only one contrasting element between the two or more contrasting elements (11) from the operative position to the released position.

In a 64^{th} aspect according to any one of the preceding aspects, a rotation of the rotor (10), when the pump (1) is in the released configuration, is prevented, in particular blocked.

In a 65^{th} aspect according to any one of the preceding aspects, a rotation of the rotor (10), when the pump (1) is in the operative configuration, is allowed.

In a 66^{th} aspect according to any one of the preceding aspects, the releasing member (31) is coupled to the stator (2) and decoupled from the rotor (10).

In a 67^{th} aspect according to any one of the preceding aspects, the releasing member (31) has one degree of freedom with respect to the stator (2), said degree of freedom being either rotation or translation.

In a 68^{th} aspect according to any one of the preceding aspects, the releasing member (31) does not rotate with the rotor (10) about the rotor axis (RA).

In a 69^{th} aspect according to any one of the preceding aspects, during a working condition of the peristaltic pump (1), in particular while the rotor (10) rotates about the rotor axis (RA), the releasing member (21) is fixed with respect to the stator (2) and the rotor (10) rotates with respect to the releasing member (31).

A 70^{th} aspect is directed to a medical apparatus comprising one or more peristaltic pumps (1) according to any one of the preceding aspects.

A 71^{st} aspect is directed to an infusion pump comprising one or more peristaltic pumps (1) according to any one of the preceding aspects.

A 72^{nd} aspect is directed to a cycler for peritoneal dialysis comprising one or more peristaltic pumps (1) according to any one of the preceding aspects.

A 73^{rd} aspect is directed to a dialysis apparatus comprising one or more peristaltic pumps (1) according to any one of the preceding aspects.

A 74^{th} aspect is directed to a dialysis apparatus comprising:
a filtration unit having a primary chamber and a secondary chamber separated by a semi-permeable membrane;
a blood circuit having a blood withdrawal line connected to an inlet of the primary chamber, and a blood return line connected to an outlet of the primary chamber, said blood circuit being configured for connection to a patient cardiovascular system;
a blood pump to circulate blood in the blood circuit;
a dialysate line connected to an outlet of the secondary chamber;
a dialysate pump active to pump fluid in the dialysate line;
one or more infusion lines to transfer a respective solution into blood;
one or more infusion pumps to pump the respective solution in said lines;
a control unit configured to control the blood pump, the dialysate pump,
the one or more infusion pumps, wherein at least one of said blood pump, dialysate pump, one or more infusion pumps are according to any one of the preceding aspects directed to the peristaltic pump.

In a 75^{th} aspect according to the preceding aspect, said blood pump is a peristaltic pump according to any one of the preceding aspects directed to the peristaltic pump.

A 76^{th} aspect is directed to a method according to any one of the preceding aspects, said method comprising the steps of:
(1003) removing the fluid line (50) from the peristaltic pump (1) when the pump is in the released configuration; and/or
(1004) installing the fluid line (50) in the peristaltic pump (1) when the pump is in the released configuration.

In a 77^{th} aspect according to any one of the preceding aspects, the step of switching the pump from the operative configuration to the released configuration causes balancing of fluid pressures across the pump, in particular wherein a fluid differential pressure between upstream and downstream tracts of the fluid line during an operative configuration is balanced, in particular wherein said differential pressure is reduced or reset to zero.

In a 78^{th} aspect according to any one of the preceding aspects, the method comprises a inactivity procedure to prevent damages to a fluid line (50) installed on the pump (1) during a prolonged period of inactivity of the pump (1), the inactivity procedure comprising a step of switching the pump (1) from the operative configuration to the released configuration or keeping the pump in the released configuration.

In a 79^{th} aspect according to any one of the preceding aspects, the inactivity procedure comprises a step of:
evaluating an inactivity time period within which the pump (1) is inactive;
comparing said inactivity time period with a inactivity time period threshold;
if the inactivity time period exceeds the inactivity time period threshold, switching the pump (1) from the operative configuration to the released configuration or keeping the pump in the released configuration, in particular wherein said inactivity time period threshold is comprised between 2 minutes and 1 hour, in particular between 5 minutes and 30 minutes.

In a 80^{th} aspect according to any one of the preceding aspects, the method comprises a releasing procedure to switch the peristaltic pump (1) from the operative configuration to the released configuration, the releasing procedure comprising at least the following steps:
arresting or rotating the rotor (10) to a predefined angular unlocking position with respect to the stator (2), the predefined angular unlocking position being defined as a position wherein at least one of the contrasting elements (11) is engageable by the releasing member (31) to switch from the operative position to the released position and vice versa;
moving the releasing member (31), in particular rotating a releasing cam (32), from the rest position to the active position to move the at least one contrasting element (11) from the operative position to the released position, said step of moving the releasing member (31) being performed manually by an operator or by a control unit (80) through an actuator;
removing the fluid line (50) from the pump (1) or installing the fluid line (50) between the at least one contrasting element (11) and the pump race (3).

In a 81^{st} aspect according to any one of the preceding aspects, the method comprises a setting procedure to switch the peristaltic pump (1) from the released configuration to the operative configuration, the setting procedure comprising at least the following steps:
arresting or rotating the rotor (10) to a predefined angular unlocking position based on the position detected by the position sensor (70);
evaluating whether the releasing member 31 is in the rest position or in the active position, if releasing member 31 is in the rest position moving the releasing member (31), in particular rotating the releasing cam (32), from the rest position to the active position to move the at least one contrasting element (11) from the operative position to the released position, said step of moving the releasing member (31) being performed manually by an operator or by the control unit (80) through an actuator;
installing the fluid line (50) between the at least one contrasting element (11) and the pump race (3);
moving the releasing member (31), in particular rotating the releasing cam (32), from the active position to the rest position to move the at least one contrasting element (11) from the released position to the operative position, said step of moving the releasing member (31) being performed manually by an operator or by the control unit (80) through an actuator.

In a 82^{nd} aspect according to any one of the preceding aspects, the method comprises a substituting procedure including the steps of:
arresting or rotating the rotor (10) to a predefined angular unlocking position based on the position detected by the position sensor (70);
moving the releasing member (31), in particular rotating the releasing cam (32), from the rest position to the active position to move the at least one contrasting element (11) from the operative position to the released position, said step of moving the releasing member (31) being performed manually by an operator or by the control unit (80) through an actuator;
removing the fluid line (50) between the at least one contrasting element (11) and the pump race (3);
installing the fluid line (50) between the at least one contrasting element (11) and the pump race (3)
moving the releasing member (31), in particular rotating the releasing cam (32), from the active position to the rest position to move the at least one contrasting element (11) from the released position to the operative position, said step of moving the releasing member (31) being performed manually by an operator or by the control unit (80) through an actuator.

In a 83^{rd} aspect according to any one of the preceding aspects, the method comprises an inactivity procedure including the steps of:
arresting or rotating the rotor (10) to the predefined angular unlocking position based on the position detected by the position sensor (70);
moving the releasing member (31), in particular rotating the releasing cam (32), from the rest position to the active position to move the at least one contrasting element (11) from the operative position to the released position, said step of moving the releasing member (31) being performed manually by an operator or by the control unit (80) through an actuator;
wherein, during said prolonged inactivity periods of the pump (1), the fluid line (50) is mounted on the pump (1), in particular wherein the fluid line (50) is interposed between at least one of the contrasting element (11) and the pump race (3).

In a 84^{th} aspect according to any one of the preceding aspects, the method comprises a pressure balancing procedure including at least the steps of:
arresting, or rotating the rotor (10), at the predefined angular unlocking position based on the position detected by the position sensor (70) of the rotor (10);
moving the releasing member (31), in particular rotating the releasing cam (32), from the rest position to the active position to move the at least one contrasting element (11) from the operative position to the released position, this step of moving the releasing member (31) being performed manually by an operator or by the control unit (80) through the actuator of the releasing member (31).

In a 85^{th} aspect according to the preceding aspect, the method comprises the steps of:
receiving at least one pressure signal representative of a fluid pressure upstream and downstream the peristaltic pump (1) or a signal representative of a differential pressure across the pump (1), in particular a differential pressure between an upstream section of the fluid line and a downstream section of the fluid line;
determining a differential pressure value representative of a differential pressure across the pump (1);
comparing said differential pressure value with at least one respective threshold value;
if said differential pressure value exceeds the at least one respective threshold value, performing the balancing procedure.

### DRAWINGS

Some embodiments and some aspects of the invention will be described below with reference to the attached drawings, provided for illustrative purposes only, wherein:

FIG. 1 is a perspective view of a peristaltic pump according to the present invention with the tube line installed;

FIGS. 2 and 3 are front views of a peristaltic pump according to the present invention in an operative and released configuration respectively;

FIG. 4 is a perspective view of the rotor of a peristaltic pump according to the present invention in an operative position;

FIG. 5 is a detailed view of the peristaltic pump according to the present invention in a released configuration;

FIG. 6 is a front view of a peristaltic pump according to the present invention in a released configuration with part of the rotor unmounted;

FIG. 7 is a perspective view of a peristaltic pump according to the present invention with the rotor unmounted;

FIG. 8 is an exploded view of a peristaltic pump according to the present invention;

FIG. 9 is a flow chart representing method steps related to the peristaltic pump of the present invention.

### DEFINITIONS

In this detailed description, corresponding parts illustrated in the various figures are indicated with the same numerical references. The figures may illustrate the invention by means of non-scale representations; therefore, parts and components illustrated in the figures relating to the object of the invention may relate exclusively to schematic representations.

Upstream and/Downstream

The terms upstream and downstream refer to a direction or trajectory of advancement of a fluid configured to flow within the fluid line during standard usage of the peristaltic pump. In particular the fluid is configured to flow from an upstream tract to a downstream tract of the fluid line: the pump is interposed between the downstream and the upstream tract of the fluid line. The upstream tract is commonly known as the supply line, while the downstream tract is commonly known as the delivery line.

### DETAILED DESCRIPTION

### Peristaltic Pump

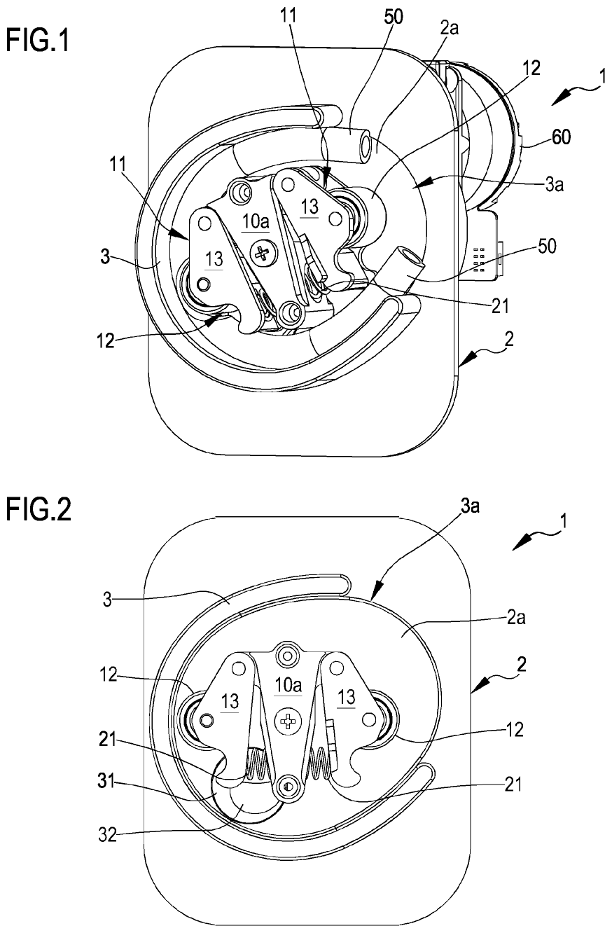

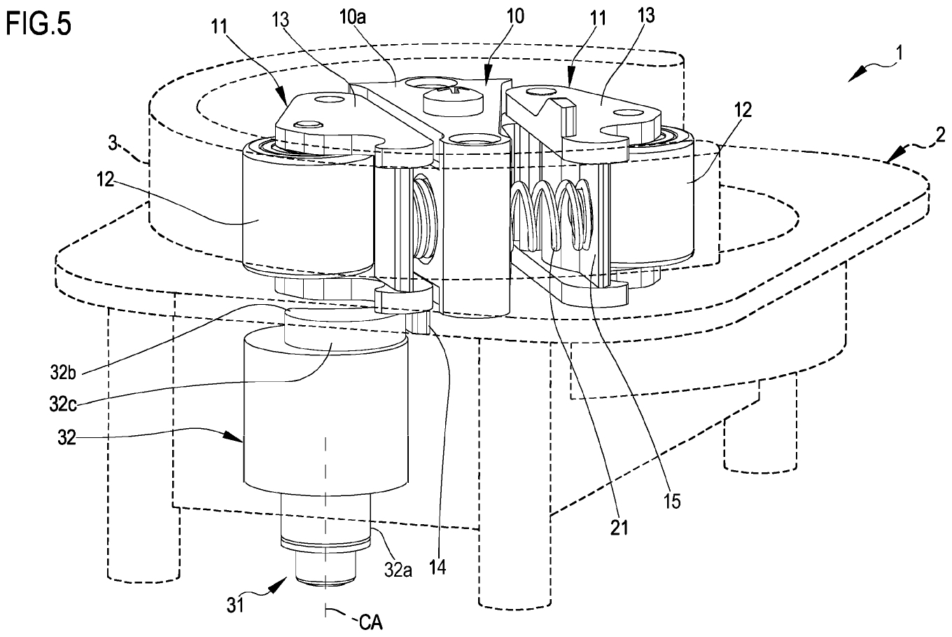

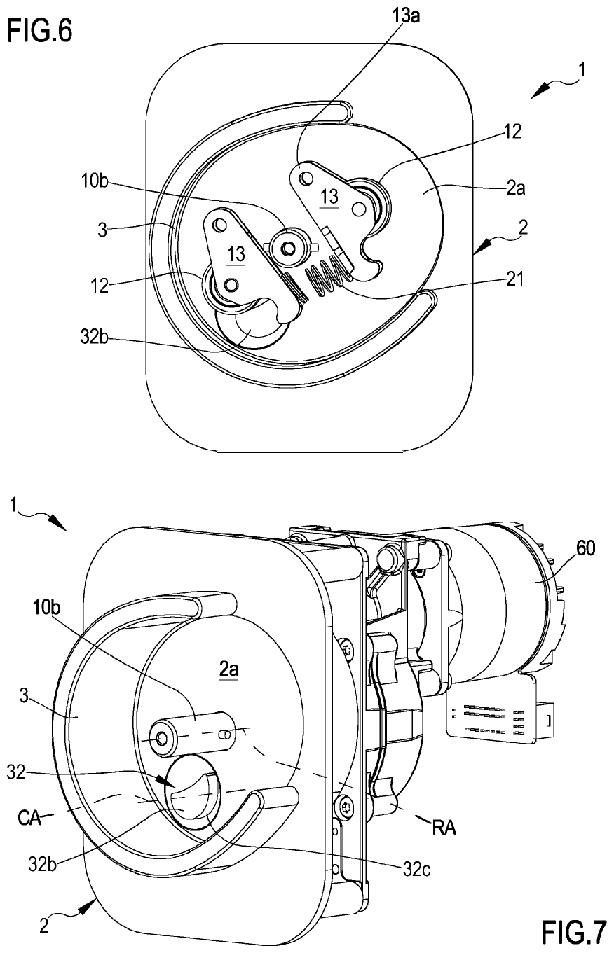

Reference number 1 is directed to a peristaltic pump as shown in FIGS. 1-8. The working principle of a peristaltic pump is well known to a skilled person: thus, the features described here after and associated to the main components of a common peristaltic pump have to be read as exemplary, as the skilled person is able to provide further modification non relating to the key concept of the present invention, which necessarily involves the presence of a releasing system 30 configured to ease installation and removal procedures of a fluid line.

The peristaltic pump of the present invention may be used for several purposes in a broad range of technical fields. In particular, the peristaltic pump of the present invention is directed to the medical field, in particular to dialysis field, wherein the fluid line may transport blood, dialysis fluid, replacement fluid, infusion fluid or the like. Normally, peristaltic pump in medical field are configured to provide a fluid rate for blood within a range between 50 ml/min and 500 ml/min, more in particular between 150 ml/min and 400 ml/min. Note that usually blood flow rate is paired with a dialysate flow of about double the blood flow rate (e.g. 100 ml/min to 1000 ml/min-300 ml/min to 800 ml/min).

Peristaltic pump 1 is configured to receive a fluid line 50 having a diameter comprised between 3 mm and 8 mm: the fluid line may be made by plastic or silicone material. The main components of the peristaltic pump 1 are the stator 2, which defines the static body of the pump, and the rotor 10 which is rotatable about a rotor axis RA: the stator 2 carries the rotor 10. An exemplary peristaltic pump 1 with a tube line installed is shown in FIG. 1.

The stator 2 comprises a base wall 2a and a pump race 3 emerging in height from the base wall 2a, defining an internal volume 5: in particular the pump race 3 emerges substantially normally with respect to the base wall 2a. The pump race 3 may have a height comprised between 3 mm and 20 mm. The base wall may have a substantially flat shape or slightly conic, while the pump race 3 may have a semicircular shape, i.e. “C” or “U” shape, concentric to the rotor axis RA defining an internal wall configured to receive in abutment the fluid line: the fluid line, when arranged in the stator 2, defines a loop as in FIG. 1. The internal wall of the pump race 3 defines an operative arc wherein the rotor 10 is configured to operatively cooperate with the fluid line to promote fluid flow within the fluid line. The internal volume 5 of the stator 2 is laterally confined by this internal wall of the pump race 3 and confined on the bottom by the base wall 2a of the stator 2.

The pump race 3 also has a lateral aperture 3a, as shown in FIGS. 1 and 2, configured to allow an inlet tract of the fluid line 50 to enter in the inner volume of the pump, and an exit tract of the fluid line to exit the pump. The lateral aperture defines a non-operative arc wherein the fluid line does not cooperate with the rotor: The non-operative arc may have an angular dimension comprised between 20° and 200°, more in particular between 450 and 180°, more in particular between 60° and 135°.

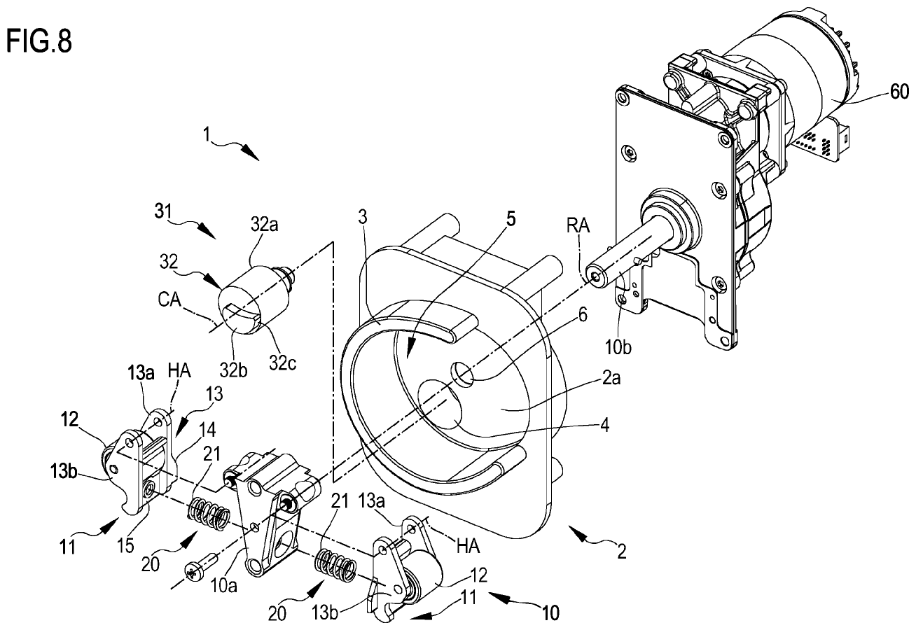

The stator 2 also has a through aperture 6 defining an aperture axis AA coincident with the rotor axis RA: the through aperture 6 is configured to bear a rotor axle 10b of the rotor 10, as shown in the exploded view of FIG. 8.

The stator 2 may be made by plastic or metallic material.

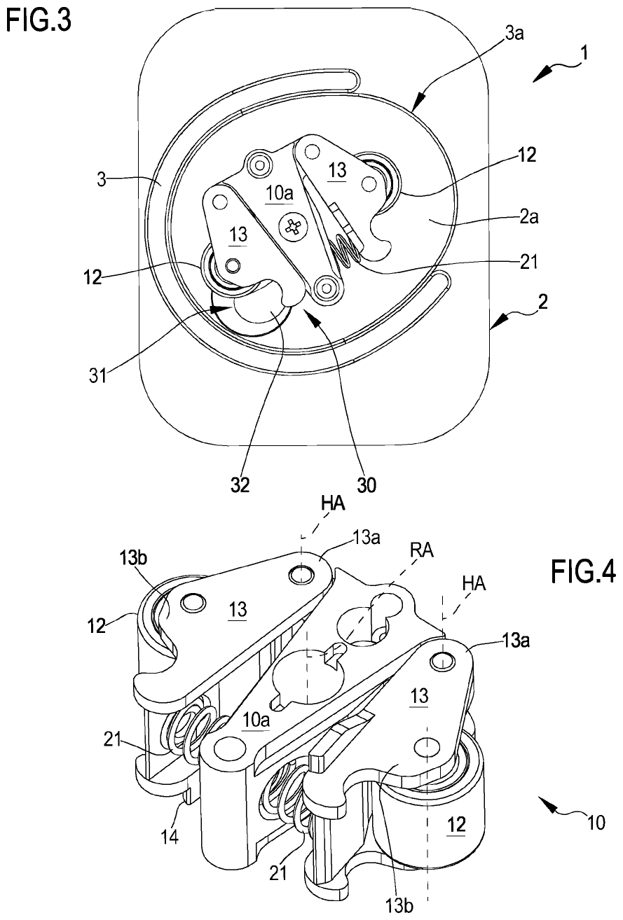

The rotor 10 of the present peristaltic pump 1 is surrounded at least partially by the pump race 3 and configured to rotate about the rotor axis RA. The rotor 10 is arranged at least partially in the internal volume 5 of the stator and may include a rotor axle 10b, carried by the stator and arranged in the through aperture 6 of the stator, and a rotor plate 10a coupled or coupleable to the rotor axle. The rotor 10 includes at least one contrasting element 11, carried by the rotor plate 10a, configured to abut against the fluid line 50 and also rotatable about the rotor axis RA. The at least one contrasting element 11 is movable towards and away with respect to the rotor axis RA and the pump race 3 between a released position shown in FIGS. 3, 5 and 6, wherein the at least one contrasting element 11 is away from the pump race 3, and an operative position shown in FIGS. 1, 2, 3 wherein the at least one contrasting element 11 is approached to the pump race 3 and configured to abut against the fluid line 50. Notably, in the released position, the contrasting element 11 is at a distance from the pump race 3 higher than a corresponding distance in the operative position. In particular the contrasting element 11, when arranged in the released position, defines a distance with respect to the inner wall of the pump race 3 comprised between 0.7 and two times an external diameter of the fluid line, more in particular between one and 1.5 times an external diameter of the fluid line. More in detail, this distance is comprised between 3 mm and 8 mm. In other terms, a contrasting element 11, when arranged in the released position, is configured to reduce or completely remove a compression force on the fluid line 50 when the latter is installed on the pump 1.

On the other hand, the contrasting element 11, when arranged in the operative position, defines a distance with respect to the inner wall of the pump race 3 comprised between 2 mm and 3 mm, in order to compress the fluid line 50 against the pump race.

In an embodiment, the peristaltic pump 1 comprises two or more contrasting elements 11: in particular the contrasting elements 11 may be in a number comprised between two and ten, more in particular between two and six according to design requirements.

Each contrasting element 11 may include at least one roller or shoe 12 configured to contact and squeeze the fluid line 50 against the pump race 3, as shown in FIG. 1. FIG. 1 show an embodiment of the pump using rollers 12 in order to reduce tangential stresses on the fluid line.

Each contrasting element 11 comprises a lever arm 13 extending in length between a hinged end 13a and a free end 13b, wherein the hinged end 13a is hinged to the rotor plate 10a, and the free end 13b carries the roller or shoe 12. The lever arm 13 is rotatable about the hinged end 13a about a hinge axis HA and with respect to the rotor plate 10a: a rotation of the lever arm 13 about the hinge axis HA determines the passage of the respective contrasting element 11 between the operative position and the released position and vice versa.

In an embodiment wherein the pump comprises two contrasting elements 11, the two contrasting elements 11 are arranged diametrically opposite each other, namely defining a 1800 angle in between. In a further embodiment wherein the pump comprises three or more contrasting elements 11, the contrasting elements 11 may be angularly spaced each other in a substantially evenly manner, in particular wherein the three or more contrasting elements are equally spaced each other.

The lever arm 13 has an extended body and may have an abutment protrusion 14 emerging from the extended body: the abutment protrusion 14 is interposed between the extended body and the base wall 2a of the stator 2. The abutment protrusion 14 may emerge from the extended body of the lever arm 13 towards the base wall 2a of the stator 2, so that the abutment protrusion 14 is the closest portion of the lever arm 13 to the base wall 2a of the stator 2. In other terms, the abutment protrusion 14 reduces a distance between the lever arm 13 and the base wall 2a of the stator 2: the distance between the abutment protrusion 14 and the base wall 2a may be comprised between 1.5 mm and 10 or more mm.

In an embodiment, only one contrasting element 11 of the two or more contrasting elements comprises the abutment protrusion 14 interposed between the extended body and the base wall 2a of the stator 2 and engageable by a releasing member 31 described here after. In a further embodiment, two contrasting elements 11 of the two or more contrasting elements comprise respective abutment protrusions 14 interposed between the extended body and the base wall 2a of the stator 2 and engageable by the releasing member 31. In a still further embodiment, all the contrasting elements 11 of the two or three or more contrasting elements comprise respective abutment protrusions 14 interposed between the extended body and the base wall 2a of the stator 2 and engageable by the releasing member 31.

The peristaltic pump further comprises a thrusting element 20 configured to act, at least when the contrasting elements are in the operative position, in thrust on the contrasting elements 11 in an outwardly direction, wherein the outwardly direction is directed towards the pump race 3 and away from the rotor axis RA. In the embodiment shown in the attached figures, the thrusting element 20 is configured to act in thrust on the contrasting elements 11 both when the contrasting elements are in the operative position and when the contrasting elements are in the released position.

As shown in the embodiment, the pump 1 may comprise one thrusting element 20 active on two or three or more contrasting elements simultaneously. Alternatively, the pump 1 may comprise one thrusting element 20 for each contrasting elements on which the thrusting element 20 is active.

The thrusting element 20 may comprise an elastic element 21, i.e. a spring, a rubber-like element, a compressive spring or a traction spring, acting on the contrasting elements 11 as shown in FIGS. 2-6. In particular one elastic element 21 may be provided for each contrasting element. The elastic element 21 is configured to move the contrasting element 11 in the outwardly direction allowing, and in particular causing, movement of the contrasting elements 11 from the released position to the operative position. Notably the elastic element 21, although it provides a thrust on the contrasting elements 11 directed towards the pump race causing the movement of the contrasting elements 11 from the released position to the operative position, also allows the movement of the contrasting elements 11 from the operative position to the released position.

Notably, the elastic element is configured to exert, when the pump 1 is in the operative configuration, a force on the contrasting elements 11 such that the latter is able to adequately compress, in particular deform, the fluid line 50 to promote fluid flow. Furthermore, the elastic element is also configured to deform itself to allow the at least one contrasting element 11 to move in the opposite direction, namely from the operative position to the released position.

The elastic element may be a compression spring: in this case the compression spring acts on the lever arm 13 at a thrusting seat 15, wherein this thrusting seat 15 may be interposed between the hinged end 13a and the free end 13b of the lever arm 13, defining a third class lever wherein the compression spring defines the applied force and the fluid line defines the resistance force applied on the free end 13b of the contrasting element 11 through a roller. In this third class lever, the thrusting seat 15 is positioned at a distance with respect to the hinge axis HA of the lever arm 13 lower than a corresponding distance between free end 13b carrying the roller 12 and the hinge axis HA.

Alternatively, the free end 13b carrying the roller 12 may be interposed between the hinged end 13a and the thrusting seat 15, defining a second class lever wherein the compression spring defines the applied force and the fluid line defines the resistance force applied on the free end 13b of the contrasting element 11 through a roller. In this second class lever, the thrusting seat 15 is positioned at a distance with respect to the hinge axis HA of the lever arm 13 higher than a corresponding distance between free end 13b carrying the roller 12 and the hinge axis HA.

Alternatively, the elastic element may be a traction spring acting on a pulling portion of the lever arm 13, so that the hinged end 13a is interposed between the pulling portion and the free end 13b carrying the roller, defining a first class lever.

Thus, according to an embodiment wherein the pump comprises the elastic element 21, namely a spring or a rubber-like element, the elastic element is the member in charge of providing a thrusting force, through the contrasting elements 11, on the fluid line 50: this provides a flexible coupling between the rollers 12 and the fluid line, being the elastic element able to eventually compensate variations in the line thickness, stiffness or presence of impurities. Furthermore, the spring rate or the rubber-like element stiffness is set in order to be able to compress the fluid line 50 adequately and determine a fluid flow.

According to a further embodiment, the elastic element may be replaced by a thrusting cam 22 movable by rotation between a thrusting position and a non-thrusting position. In the thrusting position the thrusting cam 22 abuts against the contrasting elements 11 causing the contrasting elements 11 to move in the operative position, while in the non-thrusting position the thrusting cam 22 allows the contrasting element 11 to move in the released position. In addition, the thrusting cam 22, when arranged in the thrusting position, prevents, in particular does not allow, the contrasting elements 11 to move from the operative position to the released position. Notably, in the operative configuration of the pump 1, the thrusting cam 22 is arranged in the thrusting position, while in the released configuration the thrusting cam 22 is arranged in the non-thrusting position.

The peristaltic pump of the present invention also comprises a releasing system 30 comprising a releasing member 31 borne by the stator 2 and movable between an active position and a rest position with respect to the stator 2. In particular the releasing member 31 is engaged to the stator 2 keeping at least one degree of freedom with respect to the stator 2 to shift between the active position and the rest position, i.e. rotation about an own axis or a translation movement. The releasing member 31 of the releasing system 30 is configured, upon movement from the rest position to the active position, to shift the contrasting elements or one of the contrasting elements from the operative position to the released position.

Notably, the releasing member 31 is coupled to the stator 2 and decoupled from the rotor 10. This means that the releasing member 31 does not rotate with the rotor 10 about the rotor axis RA. According to a specific embodiment, the releasing member 31 has only one degree of freedom with respect to the stator 2, namely either rotation or translation.

In particular, during a working condition of the peristaltic pump 1, namely while the rotor 10 rotates about the rotor axis RA, the releasing member 31 is fixed with respect to the stator.

The released position of the contrasting element 11 makes installation and removal procedures of fluid line easier, as the fluid line is not compressed between the pump race 3 and the rollers 12, and also safer, as the fluid line may be removed or installed without forcing it. Furthermore, during time periods wherein the fluid line 50 is installed and the peristaltic pump 1 is not working, the releasing system 30 allows to arrange the constraining element 11 in the released position in order not to stress or damage the fluid line at the contact point. In addition, as also described after in the present description, releasing the contrasting elements 11 allows to re-establish fluid pressure balance between a delivery tract of the fluid line and a supply tract of the fluid line: in other words, this allows to re-establish fluid pressure balance upstream and downstream the peristaltic pump 1.

According to the present invention, the releasing member 31 is spatially shifted with respect to the rotor axis RA and borne by the stator 2: in particular the releasing member 31 emerges in height from the base wall 2a of the stator between the rotor axis RA and the pump race 3 within the internal volume 5 of the stator 2. This feature combination provides benefits to the releasing system of the pump, as the manufacturing process is easier and cheap, and because the releasing member 31 does not affect the rotation of the rotor during a standard working condition of the peristaltic pump. Indeed, in standard working condition, the releasing member 31 is completely decoupled from the rotor movement, thereby improving safety and reliability of the pump.

The peristaltic pump 1 is configurable at least in an operative configuration and a released configuration. In the operative configuration, the releasing member 31 is arranged in the rest position to allow the at least one contrasting element 11 to be arranged in the operative position, and the contrasting element 11 is thrusted by the thrusting element 20 in the outwardly direction towards the pump race 3. On the contrary, in the released configuration, the releasing member 31 is arranged in the active position engaging the contrasting element 11 and moving the contrasting element 11 in the released position away from the pump race 3. Thus, in the operative configuration, the contrasting elements 11 are able to compress the fluid line to cause fluid flow upon rotation of the rotor 10, while in the released configuration the contrasting element 11 is moved away from the pump race, and thus from the fluid line, to allow removal or installation of the fluid line. Notably, in the operative configuration the rotor 10 is configured to rotate about the rotor axis RA to determine a fluid flow: on the contrary, in the released configuration, rotation of the rotor 10 is prevented, in particular the rotation of the rotor is blocked so that the contrasting elements 11 are fixed in position.

In an embodiment not shown in the attached figures, the releasing member 31 may be a translating member movable by translation, i.e. the releasing member may be a lever movable between the active and the rest position, wherein in the active position the lever abuts against one or more of the contrasting elements 11 to move the latter in the released position. The lever may be moved manually by an operator or automatically by an actuator, for example an electric actuator controlled by a control unit 80.

In a further embodiment, the releasing member 31 is a releasing cam 32 rotatable about a cam axis CA: in FIG. 7 the lever arms 13, the rotor plate 10a and the rollers 12 have been hidden to show in detail the releasing cam 32.

The cam axis CA is spatially shifted with respect to the rotor axis RA: in particular the cam axis CA of the releasing cam 32 is fixed with respect to the stator 2 and the releasing cam 32 is configured to rotate about the cam axis relative to the stator 2. The cam axis CA may be substantially parallel to the rotor axis RA. A distance between the cam axis CA and the rotor axis RA may be comprised between 10 mm and 60 mm. Anyhow, the distance between the cam axis CA and the rotor axis RA is lower than a distance between the rotor axis RA and the internal wall of the pump race.

Upon rotation of the releasing cam 32, the pump 1 may be configured in the operative configuration or in the released configuration. In more detail, when the pump 1 is in the operative configuration, the releasing cam 32 is rotated in the rest position to allow the at least one contrasting element 11 to move in the operative position: the contrasting element 11 is thrusted by the thrusting element 20 in the outwardly direction. On the contrary, when the pump 1 is in the released configuration, the releasing cam 32 is rotated in the active position, wherein the releasing cam 32 engages the contrasting element 11 and moves the contrasting element 11 in the released position away from the pump race 3.

The releasing cam 32 includes a bearing axle 32a rotatable about the cam axis CA, and a cam-shaped top element 32b fixed at an end of to the bearing axle 32a, as shown in FIGS. 7 and 8. The top element 32b and the bearing axle 32a define the releasing cam 32 as a single body. Furthermore, the base wall 2a of the stator 2 comprises a through hole 4 carrying the bearing axle 32a of the releasing cam 32 as shown in the exploded view of FIG. 8.

The cam-shaped top element 32b of the releasing cam 32 emerges in height from said base wall 2a of the stator 2 (see FIG. 7) into the internal volume 5 of the stator defining a cam-shaped lateral abutment wall 32c. This height may be comprised between 1 mm and 10 mm. The cam-shaped lateral abutment wall 32c defines the thickness of the cam-shaped top element 32b that emerges from to the base wall 2a of the stator, so that the cam-shaped top element 32b is able to cooperate with one or more of the contrasting elements 11. In more detail, when the releasing cam 32 is in the active position, the cam-shaped lateral abutment wall 32c abuts against the at least one contrasting element 11 of the rotor 10, in particular against the abutment portion 14 of the lever arm 13 of the at least one contrasting element 11, to define the released configuration of the peristaltic pump. On the contrary, when the releasing cam 32 is in the rest position, the cam-shaped lateral abutment wall 32c allows the at least one contrasting element 11 to move in the operative position to define the operative configuration of the peristaltic pump.

The releasing member 31 is interposed between the rotor axis RA and the pump race 3 with respect to a radial direction normal to the rotor axis RA. In particular the releasing cam 32, and more in particular the cam axis CA of the releasing cam 32, is interposed between the rotor axis RA and the pump race 3 with respect to a radial direction normal to the rotor axis RA. In addition, the releasing member 31 may be closer to the pump race 3 than to the rotor axis RA.

According to an embodiment, when the peristaltic pump 1 comprises two contrasting elements 11, the releasing member 31, namely the releasing cam 32 or the lever, is configured to engage only one contrasting element at a time of the two or more contrasting elements 11. In other words, the releasing member 31 is configured to act on one single contrasting element 11 at a time, in particular on the contrasting element 11 positioned in the operative arc of the stator 2. The other contrasting element 11 is not engaged by the releasing member and positioned in the non-operative tract of the stator 2, as the two contrasting elements are diametrically opposite each other.

Alternatively, the peristaltic pump 1 may comprise three or more contrasting elements 11: in this case the releasing member 31 is configured to engage either one contrasting element at a time of the three contrasting elements 11, or two contrasting elements at a time of the three contrasting elements 11.

As a general rule, the releasing member 31 may be configured to act only on the contrasting elements 11 which are positioned in the operative arc of the stator 2: indeed, the other contrasting element positioned in the non-operative arc of the stator does not cooperate with the fluid line, making a retraction of these contrasting elements not necessary. In other terms, given at least one first contrasting element positioned in the operative angular arch, this at least one first contrasting element is engageable by the releasing member 31. On the contrary, given at least one second contrasting element positioned in the non-operative angular arch, the at least one second contrasting element is not engageable by the releasing member 31. Thus, the releasing member 31, when passing from the rest position to the active position, is configured to engage the at least one first contrasting element to move the latter in the released position: on the contrary, the releasing member 31, when passing from the rest position to the active position, is configured not to engage the at least one second contrasting element.

In an embodiment according to the preceding description, when switching between the operative configuration and the released configuration, the rotor 10 is arranged in a predefined angular unlocking position: the predefined angular unlocking position is defined as a position wherein at least one of the contrasting elements 11 is engageable by the releasing member 31 to switch from the operative position to the released position and vice versa. More specifically, when the rotor is in the predefined angular unlocking position, only one contrasting element 11 between the two or more contrasting elements 11 is engageable by the releasing member 31.

Notably, when the rotor is in the predefined angular unlocking position, the releasing member 31 is configured to engage all the contrasting elements 11 positioned in the operative arc to move them between the released position and the operative position, while the contrasting elements in the non-operative arc may be not engageable by the releasing member 31.

In the predefined angular unlocking position, the cam-shaped lateral abutment wall 32c of the releasing cam 32 faces the abutment protrusion 14 of the lever arm 13 of one respective contrasting element 11. In particular, the releasing cam 32, when the rotor is in the predefined angular unlocking position, is interposed between the abutment protrusion 14 of the lever arm 13 and the pump race 3 with respect to a radial direction, in particular with respect to a direction normal to the rotor axis RA.

### Drives and Control Unit

The peristaltic pump 1 may also comprise a drive motor, such as an electric motor, connected to the rotor 10 and configured to cause rotation of the rotor about the rotor axis RA and promote fluid flow within the fluid line 50. Furthermore, a position sensor 70 may be provided and configured to emit a signal representative of an angular position of the rotor 10 with respect to the stator 2. In particular the position sensor 70 of the rotor is a Hall sensor configured to detect the position of a roller or shoe 12 or a passage of a roller or shoe while the rotor 10 rotates.

In addition, an actuator, i.e. an electric actuator or motor, may be provided and connected to the releasing member 31 and configured to move the releasing member between the active position and the rest position. In particular, in case the releasing cam 32 is provided, the actuator is configured to rotate the releasing cam 32 between the active position and the rest position. Furthermore, a position sensor 71 may be provided and configured to emit a signal representative of the active position or the rest position of the releasing member 31.

The peristaltic pump may comprise a control unit 80 connected to at least one between the drive member 40, the position sensor 70 of the rotor 10, the actuator of the releasing member 31 and the position sensor of the releasing member 31.

When the control unit is connected at least to the drive member 40 and the position sensor 70 of the rotor 10, the control unit 80 is configured to receive the signal emitted by the position sensor 70 of the rotor 10, and to determine an angular position of the rotor with respect to the stator 2.

Analogously, when the control unit is connected at least to the actuator of the releasing member 31 and the corresponding position sensor 71 of the releasing member 31, the control unit 80 is configured to receive the signal emitted by the position sensor of the releasing member 31, and selectively determine whether the releasing member is in the active position or in the rest position.

The control unit may be configured to switch the peristaltic pump 1 from the operative configuration to the released configuration by performing at least the following steps:
arresting or rotating the rotor 10 at a predefined angular unlocking position with respect to the stator 2, the predefined angular unlocking position being defined as a position wherein at least one of the contrasting elements 11 is engageable by the releasing member 31 to switch from the operative position to the released position and vice versa;
moving the releasing member 31, in particular rotating a releasing cam 32, from the rest position to the active position to move the at least one contrasting element 11 from the operative position to the released position, said step of moving the releasing member 31 being performed manually by an operator or by a control unit 80 through an actuator;

and wherein the control unit 80 is configured to switch the peristaltic pump 1 from the released configuration to the operative configuration by performing at least the following steps:
arresting or rotating the rotor 10 at a predefined angular unlocking position with respect to the stator 2, the predefined angular unlocking position being defined as a position wherein at least one of the contrasting elements 11 is engageable by the releasing member 31 to switch from the operative position to the released position and vice versa;
moving the releasing member 31, in particular rotating the releasing cam 32, from the active position to the rest position to move the at least one contrasting element 11 from the released position to the operative position, said step of moving the releasing member 31 being performed manually by an operator or by the control unit 80 through an actuator.

The control unit may be also configured to perform a substituting procedure to replace a fluid line 50 in the peristaltic pump 1 by switching the pump from the operative configuration to the released configuration, removing the old fluid line, installing a new fluid line, and subsequently switching the pump from the released configuration to the operative configuration. In particular the substituting procedure comprises at least the steps of:
arresting, or rotating the rotor 10, at the predefined angular unlocking position based on the position detected by the position sensor 70 of the rotor 10;
moving the releasing member 31, in particular rotating the releasing cam 32, from the rest position to the active position to move the at least one contrasting element 11 from the operative position to the released position, this step of moving the releasing member 31 may be performed manually by an operator or by the control unit 80 through the actuator of the releasing member 31;
removing the fluid line 50 between the at least one contrasting element 11 and the pump race 3, in particular removing the fluid line 50 between the contrasting element 11 in the operative arc and the pump race 3;
installing the fluid line 50 between the at least one contrasting element 11 and the pump race 3;
moving the releasing member 31, in particular rotating the releasing cam 32, from the active position to the rest position to move the at least one contrasting element 11 from the released position to the operative position, said step of moving the releasing member 31 being performed manually by an operator or by the control unit 80 through an actuator.

The control unit 80 may also be configured to perform a releasing procedure and an installing procedure, wherein the releasing procedure is configured to remove the fluid line from the peristaltic pump 1, and the installing procedure is configured to install the fluid line in the peristaltic pump 1. Combination of the releasing procedure with the installing procedure results in the substituting procedure.

The releasing procedure comprises at least the steps of:
arresting, or rotating the rotor 10, at the predefined angular unlocking position based on the position detected by the position sensor 70 of the rotor 10;
moving the releasing member 31, in particular rotating the releasing cam 32, from the rest position to the active position to move the at least one contrasting element 11 from the operative position to the released position, this step of moving the releasing member 31 may be performed manually by an operator or by the control unit 80 through the actuator of the releasing member 31;
removing the fluid line 50 between the at least one contrasting element 11 and the pump race 3, in particular removing the fluid line 50 between the contrasting element 11 in the operative arc and the pump race 3.

The setting procedure comprises at least the steps of:
arresting, or rotating the rotor 10, at the predefined angular unlocking position based on the position detected by the position sensor 70 of the rotor 10;
moving the releasing member 31, in particular rotating the releasing cam 32, from the rest position to the active position to move the at least one contrasting element 11 from the operative position to the released position, this step of moving the releasing member 31 may be performed manually by an operator or by the control unit 80 through the actuator of the releasing member 31;
installing the fluid line 50 between the at least one contrasting element 11 and the pump race 3;
moving the releasing member 31, in particular rotating the releasing cam 32, from the active position to the rest position to move the at least one contrasting element 11 from the released position to the operative position, said step of moving the releasing member 31 being performed manually by an operator or by the control unit 80 through an actuator.

Notably, the step of arresting or rotating the rotor 10, in the setting, releasing and/or substituting procedure, may comprise the step of commanding the drive motor 60 to position the rotor 10 at the predefined angular unlocking position.

As already mentioned in description, the peristaltic pump of the present invention may allow to balance fluid pressures downstream and upstream the peristaltic pump 1 or periodically lower a differential pressure between downstream and upstream sections of the fluid line with respect to the peristaltic pump 1.

A contrasting element 11 in the operative position abuts against the fluid line at a contact point causing the fluid line to be compressed, thereby limiting or inhibiting fluid communication across the contact point between fluid located upstream the pump and fluid located downstream the pump. Shifting the contrasting element 11 from its operative position to released position allows the fluid line 50 to re-expand at contact point, causing the fluid located upstream the pump 1 to be in fluid communication with the fluid located downstream the pump 1, thereby eventually rebalancing a differential pressure.

The control unit 80 is configured to perform a pressure balancing procedure to re-balance fluid pressures across the pump, wherein the pressure balancing procedure comprises switching the pump from the operative configuration to the released configuration. In particular the balancing procedure may comprise the steps of:
arresting, or rotating the rotor 10, at the predefined angular unlocking position based on the position detected by the position sensor 70 of the rotor 10;
moving the releasing member 31, in particular rotating the releasing cam 32, from the rest position to the active position to move the at least one contrasting element 11 from the operative position to the released position, this step of moving the releasing member 31 may be performed manually by an operator or by the control unit 80 through the actuator of the releasing member 31.

Furthermore, the control unit 80 may be configured to:
receive at least a pressure signal representative of a fluid pressure upstream and downstream the peristaltic pump 1 or a signal representative of a differential pressure across the pump, in particular a differential pressure between an upstream section of the fluid line and a downstream section of the fluid line;
determine a differential pressure value representative of a differential pressure across the pump 1;
compare the differential pressure value with at least one respective threshold value;
if the differential pressure value exceeds the at least one respective threshold value, perform the balancing procedure.

According to an embodiment, the balancing procedure is performed during an extracorporeal blood treatment, in particular wherein the fluid in the fluid line 50 is blood.

As already mentioned in the description, a prolonged compression of one roller 12 on the fluid line during a period of inactivity of the pump 1, namely during a period wherein the rotor 10 does not rotate, may lead to damage the fluid line at the contact point with the roller. For example, a prolonged compression may lead to plastic deformation of the fluid line.

In order to avoid this issue, the pump 1 of the present invention allows to retract the roller during inactivity periods of the pump 1, so that no compression or a reduced compression is applied on the fluid line by the roller. On this regard, the control unit 80 is configured to perform a inactivity procedure to prevent damage to the fluid line 50 during prolonged inactivity periods of the pump 1. The inactivity procedure comprises switching the pump from the operative configuration to the released configuration. In particular the inactivity procedure comprises the steps of:
arresting or rotating the rotor 10 at the predefined angular unlocking position based on the position detected by the position sensor 70;
moving the releasing member 31, in particular rotating the releasing cam 32, from the rest position to the active position to move the at least one contrasting element 11 from the operative position to the released position, said step of moving the releasing member 31 being performed manually by an operator or by the control unit 80 through an actuator.

During the prolonged inactivity periods of the pump 1, the fluid line 50 is mounted on the pump 1 interposed between the contrasting elements 11 and the pump race 3.

The inactivity procedure may be triggered by an operator through a user interface, such as a graphical user interface or a button, operatively connected to the control unit 80. Alternatively, the control unit may be configured to:
evaluate an inactivity time period within which the pump is inactive;
compare said inactivity time period with a respective inactivity time period threshold;
if the inactivity time period exceeds the inactivity time period threshold, trigger the inactivity procedure.

The threshold period may be received as input by a user by a user interface or may be a predefined value. The inactivity time period threshold may be comprised between 2 minutes and 1 hour, in particular between 5 minutes and 30 minutes.

The control unit may be also configured to perform safety procedures aimed to prevent damages to the pump and increase reliability of the pump. According to this aim, the control unit 80 may be configured to:
detect the position of the rotor 10 by the position sensor 70;
evaluate whether said detected position of the rotor 10 is coincident with the predefined angular unlocking position;
if the detected position of the rotor 10 is coincident with the predefined angular unlocking position, allow the actuator of the releasing member 31 to move the releasing member 31 from the rest position to the active position;
if the detected position of the rotor 10 does not coincide with the predefined angular unlocking position, prevent the actuator of the releasing member 31 to move the releasing member 31 from the rest position to the active position.

Furthermore, the control unit 80 may be configured to:
detect the position of the releasing member 31 by the position sensor 71 of the releasing system 30;
evaluate whether this detected position of the releasing member 31 corresponds to the active position or the rest position;
if the releasing member 31 is detected to be in the active position, prevent rotation of the rotor 10 by inhibiting rotation of the drive motor 60 of the rotor 10.

The step of preventing rotation of the drive motor 60 may be performed by the control unit by preventing the drive motor 60 to be supplied by an electrical current.

As a general, a rotation of the rotor 10, when the pump 1 is in the released configuration, is prevented, in particular blocked. On the other hand, when the pump 1 is in the operative configuration, a rotation of the rotor 10 is allowed.

### Methods

The present disclosure is also directed to methods performed by the peristatic pump 1 according to the preceding description and to the attached claims or aspects.

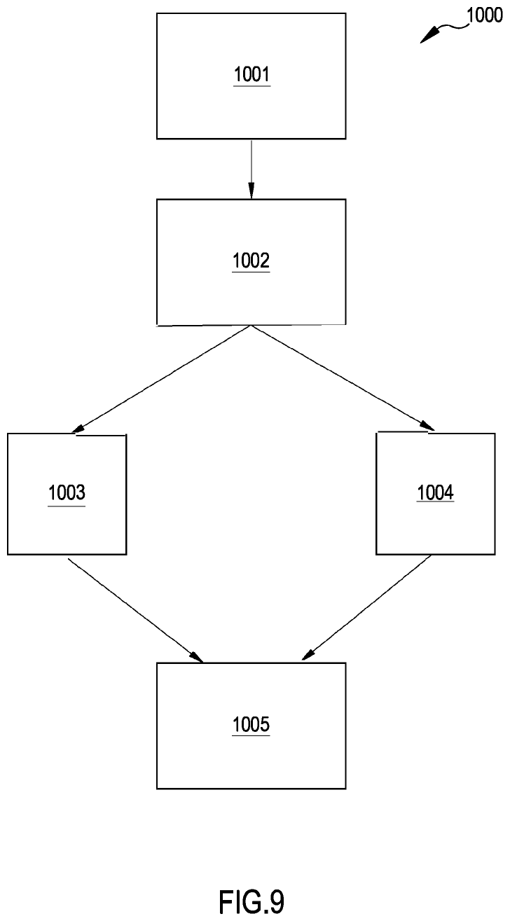

A method 1000, shown in the flow chart of FIG. 9, is provided to switch the peristaltic pump 1 from the operative configuration to the released configuration and vice versa. The method to switch the peristaltic pump 1 from the operative configuration to the released configuration comprises:
a step 1001 including arresting or rotating the rotor 10 at a predefined angular unlocking position with respect to the stator 2, the predefined angular unlocking position being defined as a position wherein at least one of the contrasting elements 11 is engageable by the releasing member 31 to switch from the operative position to the released position and vice versa;
a step 1002 including moving the releasing member 31, in particular rotating a releasing cam 32, from the rest position to the active position to move the at least one contrasting element 11 from the operative position to the released position, said step of moving the releasing member 31 being performed manually by an operator or by a control unit 80 through an actuator

The method to switch the peristaltic pump 1 from the released configuration to the operative configuration comprises at least:
arresting or rotating the rotor 10 at a predefined angular unlocking position with respect to the stator 2, the predefined angular unlocking position being defined as a position wherein at least one of the contrasting elements 11 is engageable by the releasing member 31 to switch from the operative position to the released position and vice versa;
a step 1005 including moving the releasing member 31, in particular rotating the releasing cam 32, from the active position to the rest position to move the at least one contrasting element 11 from the released position to the operative position, said step of moving the releasing member 31 being performed manually by an operator or by the control unit 80 through an actuator.

In addition, the method may comprise:
a step 1003 including removing the fluid line 50 from the peristaltic pump 1 when the pump is in the released configuration, and/or
a step 1004 including installing the fluid line 50 in the peristaltic pump 1 when the pump is in the released configuration.

Notably, the step of switching the pump from the operative configuration to the released configuration causes balancing of fluid pressures across the pump, in particular wherein a fluid differential pressure between upstream and downstream tracts of the fluid line during an operative configuration is balanced. In particular the differential pressure is reduced or reset to zero when the pump is arranged in the released configuration as the fluid upstream the pump is in fluid communication with the fluid downstream the pump.

The method may also comprise an inactivity procedure to prevent damages to a fluid line 50 installed on the pump 1 during a prolonged period of inactivity of the pump 1: the inactivity procedure comprises a step of switching the pump 1 from the operative configuration to the released configuration or keeping the pump in the released configuration. This allows to remove compression provided by the roller 12 on the fluid line when the contrasting element 11 is in the operative position, namely when the roller squeezes the fluid line.

Furthermore, the inactivity procedure may comprise:
evaluating an inactivity time period within which the pump (1) is inactive;
comparing said inactivity time period with a inactivity time period threshold;
if the inactivity time period exceeds the inactivity time period threshold, switching the pump (1) from the operative configuration to the released configuration or keeping the pump in the released configuration.

The inactivity time period threshold may be comprised between 2 minutes and 1 hour, in particular between 5 minutes and 30 minutes.

A method for removing a fluid line 50 in a peristaltic pump 1 may also be provided, wherein this method comprises a releasing procedure to switch the peristaltic pump 1 from the operative configuration to the released configuration, the releasing procedure comprising at least the following steps:
arresting or rotating the rotor 10 at a predefined angular unlocking position with respect to the stator 2, the predefined angular unlocking position being defined as a position wherein at least one of the contrasting elements 11 is engageable by the releasing member 31 to switch from the operative position to the released position and vice versa;
moving the releasing member 31, in particular rotating a releasing cam 32, from the rest position to the active position to move the at least one contrasting element 11 from the operative position to the released position, said step of moving the releasing member 31 being performed manually by an operator or by a control unit 80 through an actuator;
removing the fluid line 50 from the pump 1 or installing the fluid line 50 between the at least one contrasting element 11 and the pump race 3.

A method for installing a fluid line 50 in a peristaltic pump 1 is provided, the setting procedure comprising at least the following steps:
arresting or rotating the rotor 10 at a predefined angular unlocking position based on the position detected by the position sensor 70;
evaluating whether the releasing member 31 is in the rest position or in the active position, if releasing member 31 is in the rest position moving the releasing member 31, in particular rotating the releasing cam 32, from the rest position to the active position to move the at least one contrasting element 11 from the operative position to the released position, said step of moving the releasing member 31 being performed manually by an operator or by the control unit 80 through an actuator;
installing the fluid line 50 between the at least one contrasting element 11 and the pump race 3
moving the releasing member 31, in particular rotating the releasing cam 32, from the active position to the rest position to move the at least one contrasting element 11 from the released position to the operative position, said step of moving the releasing member 31 being performed manually by an operator or by the control unit 80 through an actuator.

A method for replacing a flud line 50 is a peristaltic pump 1 may be also provided, wherein the method comprises a substituting procedure including the steps of:
arresting or rotating the rotor 10 at a predefined angular unlocking position based on the position detected by the position sensor 70;
moving the releasing member 31, in particular rotating the releasing cam 32, from the rest position to the active position to move the at least one contrasting element 11 from the operative position to the released position, said step of moving the releasing member 31 being performed manually by an operator or by the control unit 80 through an actuator;
removing the fluid line 50 between the at least one contrasting element 11 and the pump race 3;
installing the fluid line 50 between the at least one contrasting element 11 and the pump race 3
moving the releasing member 31, in particular rotating the releasing cam 32, from the active position to the rest position to move the at least one contrasting element 11 from the released position to the operative position, said step of moving the releasing member 31 being performed manually by an operator or by the control unit 80 through an actuator.

A method for preventing damage to a fluid line 50 during prolonged inactivity periods of a peristaltic pump 1 may be provided, wherein this method comprises an inactivity procedure including at least the steps of:
arresting or rotating the rotor 10 at the predefined angular unlocking position based on the position detected by the position sensor 70;
moving the releasing member 31, in particular rotating the releasing cam 32, from the rest position to the active position to move the at least one contrasting element 11 from the operative position to the released position, said step of moving the releasing member 31 being performed manually by an operator or by the control unit 80 through an actuator;
wherein, during said prolonged inactivity periods of the pump 1, the fluid line 50 is mounted on the pump 1, in particular wherein the fluid line 50 is interposed between at least one of the contrasting element 11 and the pump race 3.

A method for balancing fluid pressures across a peristaltic pump 1 may be also provide, wherein the method comprises a pressure balancing procedure including at least the steps of:
arresting, or rotating the rotor 10, at the predefined angular unlocking position based on the position detected by the position sensor 70 of the rotor 10;
moving the releasing member 31, in particular rotating the releasing cam 32, from the rest position to the active position to move the at least one contrasting element 11 from the operative position to the released position, this step of moving the releasing member 31 being performed manually by an operator or by the control unit 80 through the actuator of the releasing member 31.

The balancing procedure of the previous method may also comprise the steps of:
receiving at least one pressure signal representative of a fluid pressure upstream and downstream the peristaltic pump 1 or a signal representative of a differential pressure across the pump 1, in particular a differential pressure between an upstream section of the fluid line and a downstream section of the fluid line;
determining a differential pressure value representative of a differential pressure across the pump 1;
comparing said differential pressure value with at least one respective threshold value;
if said differential pressure value exceeds the at least one respective threshold value, performing the balancing procedure.

Parts list

| | |
|---|---|
| 1 | Peristaltic pump |
| 2 | Stator |
| 3 | Pump race |
| 3a | Lateral aperture of the pump race 3 |
| 4 | through hole of the stator 2 |
| 5 | Internal volume of the stator 2 |
| 6 | through aperture of the stator 2 |
| 10 | Rotor |
| 10a | Rotor plate |
| 10b | Rotor axle |
| 11 | Contrasting element |
| 12 | Roller or shoe |
| 13 | Lever arm |
| 13a | Hinged end of the lever arm 13 |
| 13b | Free end of the lever arm 13 |
| 14 | Abutment protrusion |
| 15 | Abutment seat |
| 20 | Thrusting element |
| 21 | Elastic element |
| 22 | Thrusting cam 22 |
| 30 | Releasing system |
| 31 | Releasing member |
| 32 | Releasing cam |
| 32a | bearing axle 32a of the releasing cam 32 |
| 32b | Cam-shaped top element of the releasing cam 32 |
| 32c | Cam-shaped lateral abutment wall of the releasing cam 32 |
| 40 | Drive member |
| 50 | Fluid line |
| 60 | Drive motor of the rotor 10 |
| 70 | Position sensor of the rotor 10 |
| 71 | Position sensor of the releasing member 31 |
| 80 | Control unit |

1-15. (canceled)

16. A peristaltic pump configured to receive a fluid line, the peristaltic pump comprising:
a stator comprising a pump race;
a rotor surrounded at least partially by the pump race and configured to rotate about a rotor axis, the rotor comprising:
at least one contrasting element configured to abut against the fluid line, the at least one contrasting element being movable towards and away with respect to the rotor axis and the pump race between:
a released position where the at least one contrasting element is away from the pump race, and
an operative position where the at least one contrasting element is approached to the pump race and configured to abut against the fluid line,
wherein in the released position the at least one contrasting element is at a distance from the pump race higher than in the operative position, and
a thrusting element configured to act, at least in the operative position, in thrust on the at least one contrasting element in an outwardly direction towards the pump race; and
a releasing system comprising a releasing member borne by the stator and movable between an active position and a rest position, the releasing member being spatially shifted with respect to the rotor axis,
wherein the peristaltic pump is configurable in at least:
an operative configuration where the releasing member is moved in the rest position to allow the at least one contrasting element to move in the operative position, the at least one contrasting element being thrusted by the thrusting element in the outwardly direction, and
a released configuration where the releasing member is moved in the active position, the releasing member engaging the at least one contrasting element and moving the at least one contrasting element in the released position away from the pump race.

17. The peristaltic pump according to claim 16, wherein the releasing member is a releasing cam rotatable about a cam axis, the cam axis being spatially shifted with respect to the rotor axis, and wherein:
in the operative configuration, the releasing cam is rotated in the rest position to allow the at least one contrasting element to remain in the operative position, the at least one contrasting element being thrusted by the thrusting element in the outwardly direction; and
in the released configuration, the releasing cam is rotated in the active position, said releasing cam engaging the at least one contrasting element and maintaining the at least one contrasting element in the released position away from the pump race.

18. The peristaltic pump according to claim 16, wherein the releasing member is a translating member movable by translation along a rectilinear or curvilinear path, wherein:
in the operative configuration, the translating member is moved in the rest position to allow the at least one contrasting element to remain in the operative position, the at least one contrasting element being thrusted by the thrusting element in the outwardly direction; and
in the released configuration, the translating member is moved in the active position, the translating member engaging the at least one contrasting element and maintaining the at least one contrasting element in the released position away from the pump race.

19. The peristaltic pump according to claim 17, wherein the cam axis of the releasing cam is fixed with respect to the stator and wherein the releasing cam is configured to rotate about the cam axis relative to the stator.

20. The peristaltic pump according to claim 19, wherein the cam axis is parallel to the rotor axis.

21. The peristaltic pump according to claim 16, wherein the releasing member is interposed between the rotor axis and the pump race with respect to a radial direction normal to the rotor axis.

22. The peristaltic pump according to claim 17, wherein the releasing cam and the cam axis of the releasing cam are interposed between the rotor axis and the pump race with respect to a radial direction normal to the rotor axis.

23. The peristaltic pump according to claim 17, wherein the stator comprises a base wall, the pump race protruding in height from the base wall defining an internal volume confined in depth by the base wall and laterally by the pump race, the rotor being arranged at least partially within the internal volume of the stator,
wherein the releasing cam includes a bearing axle rotatable about the cam axis, and a cam-shaped top element fixed at an end of to the bearing axle, the bearing axle in combination with the cam-shaped top element defining the releasing cam as a single body, and
wherein the base wall of the stator comprises a through hole allowing the bearing axle of the releasing cam to pass through, the cam-shaped top element of the releasing cam emerging in height from the base wall of the stator into the internal volume defining a cam-shaped lateral abutment wall,
wherein:
when the releasing cam is in the active position, the cam-shaped lateral abutment wall abuts against the at least one contrasting element of the rotor to define the released configuration of the peristaltic pump, and
when the releasing cam is in the rest position, the cam-shaped lateral abutment wall allows the at least one contrasting element to remain in the operative position to define the operative configuration of the peristaltic pump.

24. The peristaltic pump according to claim 23, wherein the rotor comprises a rotor plate bearing the at least one contrasting element, and wherein the at least one contrasting element includes:
a roller or shoe configured to contact and squeeze the fluid line against the pump race in the operative position of the peristaltic pump; and
a lever arm extending between a hinged end and a free end, the lever arm being rotatable about the hinged end about a hinge axis, wherein:
the hinged end is hinged to the rotor plate thereby allowing rotation of the lever arm about the hinge axis, and
the free end carries the roller or shoe,
wherein a rotation of the lever arm about the hinge axis determines the passage of the respective contrasting element between the operative position and the released position, wherein a distance between the at least one contrasting element and the pump race is defined as a distance interposed between the roller or shoe and the pump race.

25. The peristaltic pump according to claim 24, wherein the lever arm comprises an extended body and an abutment protrusion emerging from the extended body, the abutment protrusion being configured to abut against the cam-shaped lateral abutment wall of the releasing cam when the releasing cam is in the active position, the abutment protrusion being interposed between the extended body and the base wall of the stator.

26. The peristaltic pump according to claim 16, wherein the pump comprises:
two or more contrasting elements angularly spaced to each other, the releasing member being configured to engage one or two contrasting elements at a time between the two or more contrasting elements.

27. The peristaltic pump according to claim 16, wherein the thrusting element comprises an elastic element acting on the at least one contrasting element and configured to move the at least one contrasting element in the outwardly direction, the elastic element causing movement of the at least one contrasting element from the released position to the operative position,
and wherein the releasing member is configured, during a passage between the operative configuration to the released configuration, to exert a force on the at least one contrasting element opposite in direction to a force exerted by the thrusting element on the at least one contrasting element.

28. The peristaltic pump according to claim 16, wherein the thrusting element comprises a thrusting cam movable by rotation between:
a thrusting position wherein the thrusting cam abuts against the at least one contrasting element causing the at least one contrasting element to move in the operative position, and
a non-thrusting position wherein the thrusting cam allows the at least one contrasting element to move in the released position.

29. The peristaltic pump according to claim 27, wherein the elastic element is configured to:
exert, when the pump is in the operative configuration, a force on the at least one contrasting element such that the latter is able to squeeze the fluid line to promote fluid flow, and
deform itself to allow the at least one contrasting element to move from the operative position to the released position.

30. The peristaltic pump according to claim 16, wherein, when switching between the operative configuration and the released configuration, the rotor is arranged in a predefined angular unlocking position,
wherein, when the rotor is in the predefined angular unlocking position, the at least one contrasting element is engageable by the releasing member to switch from the operative position to the released position and vice versa,
the pump race having:
an inner wall configured to contact the fluid line, the inner wall defining an operative angular arch for the contrasting elements, the contrasting elements facing the inner wall of the pump race, and
a lateral aperture configured to allow an inlet tract of the fluid line to be received by the pump, and an exit tract of the fluid lined to exit the pump, the lateral aperture defining a non-operative angular arch for the at least one contrasting element, the at least one contrasting element when in the non-operative angular arch is not configured to cooperate with the fluid line, and
wherein the rotor, when arranged in the predefined angular unlocking position, has:
at least one first contrasting element positioned in the operative angular arch, wherein the at least one first contrasting element is engageable by the releasing member; and
at least one second contrasting element positioned in the non-operative angular arch, wherein the at least one second contrasting element is not engageable by the releasing member,
wherein the releasing member, when arranged in the predefined angular unlocking position, is configured to engage the at least one first contrasting element to move the latter in the released position,
wherein the releasing member, when passing from the rest position to the active position, is configured not to engage the at least one second contrasting element.

31. The peristaltic pump according to claim 30, wherein the peristaltic pump comprises:
a drive motor operatively connected to the rotor and configured to rotate the rotor about the rotor axis to determine a fluid flow in the fluid line;
a position sensor configured to emit a signal representative of an angular position of the rotor with respect to the stator;
a control unit operatively connected to the drive motor and the position sensor, the control unit being configured to receive the signal emitted by the position sensor and to determine one of: an angular position of the rotor with respect to the stator and a position of at least one contrasting element with respect to the releasing member,
wherein the control unit is configured to perform a:
releasing procedure configured to switch the peristaltic pump from the operative configuration to the released configuration, the releasing procedure comprising:
rotating the rotor to, and arresting the rotor at the predefined angular unlocking position based on the position detected by the position sensor;
moving the releasing member from the rest position to the active position to move the at least one contrasting element from the operative position to the released position, the step of moving the releasing member being performed manually by an operator or by the control unit through an actuator; and
removing the fluid line between the at least one contrasting element and the pump race; and
a setting procedure, subsequent to the releasing procedure, configured to switch the peristaltic pump from the released configuration to the operative configuration, the setting procedure comprising:
rotating the rotor to, and arresting the rotor at the predefined angular unlocking position based on the position detected by the position sensor;
moving the releasing member from the rest position to the active position to move the at least one contrasting element from the operative position to the released position, the step of moving the releasing member being performed manually by an operator or by the control unit through an actuator;
installing the fluid line between the at least one contrasting element and the pump race; and
moving the releasing member from the active position to the rest position to move the at least one contrasting element from the released position to the operative position, the step of moving the releasing member being performed manually by an operator or by the control unit through an actuator.

32. The peristaltic pump according to claim 31, wherein the position sensor is a Hall sensor configured to detect a position or a passage of a roller or shoe of the at least one contrasting element.

33. The peristaltic pump according to claim 16, wherein the releasing system comprises a position sensor operatively connected to the control unit and configured to emit a signal representative of the active or rest position of the releasing member, the control unit being configured to selectively determine whether the releasing member is in the active or rest position,
the pump further comprising an actuator operatively connected to the control unit and to the releasing member, the actuator being configured to move the releasing member between the rest position and the active position upon command of the control unit,
wherein the control unit is configured to:
detect the position of the releasing member by the position sensor of the releasing system,
evaluate whether the detected position of the releasing member corresponds to the active position or the rest position, and
if the releasing member is detected to be in the active position, prevent rotation of the rotor by inhibiting rotation of the drive motor of the rotor.

34. The peristaltic pump according to claim 30, wherein the distance between the at least one contrasting element and the pump race is defined as a distance interposed between a roller or shoe of the at least one contrasting element, when arranged in the operative angular arch of the pump race, and the pump race.

35. The peristaltic pump according to claim 16, wherein the releasing member does not rotate with the rotor about the rotor axis, and wherein, during a working condition of the peristaltic pump and during a rotation of the rotor about the rotor axis, the releasing member is fixed with respect to the stator and the rotor is configured to rotate with respect to the releasing member.
